(19) United States
(12) Patent Application Publication
(10) Pub. No.: US 2012/0259303 A1
(43) Pub. Date: Oct. 11, 2012

(54) Disposable breast pad

(76) Inventor: Marcia Angela Carter, Parkland, FL (US)

(21) Appl. No.: 13/066,236

(22) Filed: Apr. 11, 2011

(51) Int. Cl. A61F 7/10; A61F 13/45

(52) U.S. Cl. 604/385.01; 607/108

(57) ABSTRACT

The present invention is referred to a nursing breast pad, comprising a circular pad including a front garment facing surface and a rear wearer facing surface attached to each other by a peripheral flange. The space between the garment facing surface and the wearer facing surface contains a liquid chemical and a powder chemical that, when mixed together, can produce an endothermic cooling action. The liquid chemical is contained within a flexible bag that, when squeezed by the user, causes the bag to burst so that the two chemicals can mix.

### FIELD OF THE INVENTION

The present invention relates generally to breast pads use by breastfeeding mothers to absorb breast milk from leaking breasts. More particularly, the present invention is directed to a disposable nursing breast pad having an inner cooling portion in contact with a nipple for increased healing effect and comfort and an outer portion for pulling the moisture away from the nipples.

### DESCRIPTION OF THE PRIOR ART

Extensive research documents diverse and compelling advantages for infants, mothers, families, and society from breastfeeding and use of human milk for infant feeding. These advantages include health, nutritional, immunologic, developmental, psychological, social, economic, and environmental benefits. The American Academy of Pediatrics (AAP) emphasized the importance of breastfeeding in an article called Breastfeeding and the Use of Human Milk.

Human milk is species-specific, and all substitute feeding preparations differ markedly from it, making human milk uniquely superior for infant feeding. The benefits of breastfeeding are countless, and only some of them may be summarized as:
a decrease in the incidence and/or severity of a wide range of infectious diseases including bacterial meningitis, diarrhea, respiratory tract infection, otitis, etc.;
decreased rates of a sudden infant death syndrome in the first year of life;
slightly enhanced performance on tests of cognitive development;
decreased postpartum bleeding and more rapid uterine involution attributable to increased concentrations of oxytocin;
earlier return to pre-pregnancy weight;
potential for decreased annual health care costs; decreased costs for public nutrition health programs, decreased parental employee absenteeism; etc.

However, not all the breastfeeding experiences are successful or pleasant, as almost every mother who attempts to breast feed her baby experiences some kind of sore and painful nipples. Several problems may occur, and for many women cracked, bleeding nipples and even breast infections may be involved. These problems are the main reason why many women abandon breastfeeding.

There are several treatments and procedures known in the prior art attempting to solve these problems. For example, a disposable nursing breast pad is known, contoured to fit the breast of a nursing woman, comprising a soft, highly absorbent material. Disposed adjacent the center of the pad's inner surface is a lubricating moisturizer, such as lanolin, to protect the woman's nipple and prevent dryness and cracking. The pads outer surface is provided with an adhesive for adhering to the inner surface of clothing, such as a regular or nursing brassiere, for securely maintaining the nursing pad in position on the woman's nipple when not nursing to absorb leaked milk and prevent staining of a garment worn by the woman.

Furthermore, an areola pad for use during breastfeeding is also known, comprising a flexible element composed of one or more bio-compatible polymers, where the element is breathable, and allows contact between the mother and the infant. The flexible element further includes a centrally located opening for receiving a nipple of any size, where the nipple extends through the opening and is uncovered.

Another known option is a breast pad having a wax-like medicament, such as lanolin, disposed on an exposed surface of an inner liner of the pad in only a limited region, leaving other regions of the inner liner with a permeable region free of the medicament.

In the prior art a disposable therapeutic breast pad for heating or cooling the female breast during nursing is also known comprising a generally disc-shaped, moisture impervious outer layer and an absorbent material disposed inwardly of the outer layer adapted to be soaked in hot or cold water so that the breast pad may be positioned adjacent the female breast with the absorbent material in direct contact therewith to heat or cool the female breast during nursing. Even though this solution involves a thermal treatment of the nipple, the procedure is cumbersome. In addition, soaking the material in cool water involves a very short and reduced effect on the nipple, and therefore, its results are unsatisfactory.

A nursing pad for absorbing milk leaking from a lactating user's nipple is also known. The nursing pad includes a water impermeable exterior layer with a center region, an outer perimeter, a concave inner face, and a convex outer face. The inner face of the exterior layer defines an inner space in which pluralities of coextensive generally hemispherical inner layers are disposed.

There are also several products for nipple treatment in the market. For example, Medela Incorporated commercializes a product called TENDER CARE HYDROGEL PADS as advanced soothing gel pads for a nipple therapy. These pads comprise a flexible plastic base on which a gel is applied. A plastic liner covers said gel surface before using it. To reduce stickiness and improve soothing, the gel pad must be passed under cool water before applying on the nipple. Prior to breastfeeding, the gel pad must be removed and placed on a clean surface such as the cover liner. After breastfeeding, the gel pad may be reapplied.

Another available nipple treatment device is a nipple shield commercialized under the brand name TheraShells by Medela, Inc. of McHenry, Ill. This product protects sore nipples from further irritation, and is a rigid, dome-shaped, multi-piece shell which overlies the areola and nipple to protect them from irritation, for example, from clothing. The shell includes holes which allow air to circulate around the nipple.

None of the above cited devices or treatments help to effectively ease pain and soreness usually involved during breastfeeding. Thus, there is a great need for a device which eases pain and soreness during breastfeeding, by a durable thermal treatment of the nipple, using simple and economical means.

Even though the above cited products address some of the needs of the market, a reliable and simple thermal treatment useful for soothing sore, burning and bleeding nipples is still desired.

### SUMMARY OF THE INVENTION

This invention is directed to a disposable breast pad for a nursing woman, which provides an increased healing effect for soothing sore nipples and at the same time improving the wearer's comfort.

It is another object of the present invention to provide a thermal treatment for the nipple of a nursing woman to prevent discomfort caused by dryness and cracking.

A further object of the present invention is to provide a disposable breast pad to be worn by a nursing woman that creates a cooling effect lasting between 20 to 30 minutes thereby stopping the bleeding and burning of the skin of the nipples.

Another aspect of the present invention provides a disposable breast pad which adheres to the inner surface of a worn garment, such as a regular or nursing brassiere, for maintaining the pad securely in a fixed position on the woman's nipple.

Furthermore, another aspect of this invention comprises a disposable therapeutic breast pad having an internal central region and chemicals which are activated when a liquid holding bag is depressed by the user.

In a further aspect of the present invention, the purposed disposable nursing breast pad stops breast leakage and provides a cooling relief to some nipples at the same time.

In summary, the present invention is referred to a nursing breast pad, comprising a circular pad, including a front garment facing surface and a rear wearer facing surface. The space between the two surfaces includes a centrally located circular cooling means. An absorbent pad is attached to the outside wall of the wearer facing surface. The Space between the two surfaces contain a chemical powder and a liquid that, when combined, produce an endothermic cooling reaction.

These and other aspects, features, and advantages of the present invention will become more readily apparent from the attached drawings and the detailed description of the preferred embodiments, which follow.

### BRIEF DESCRIPTION OF THE DRAWINGS

FIG. 1 is a perspective view of the invention 100.

FIG. 2. is an exploded view of the invention.

FIG. 3 is a side section view of the invention.

FIG. 4 is a plan view looking at the inside surface of the front garment facing surface.

FIG. 5 is a side section view showing the invention in place on a user's breast.

### DETAILED DESCRIPTION OF THE INVENTION

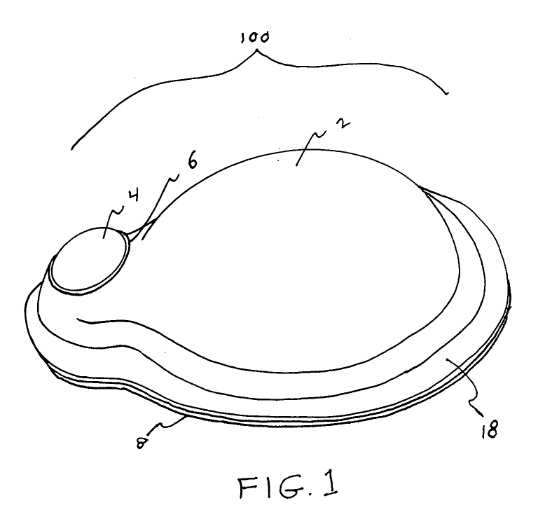

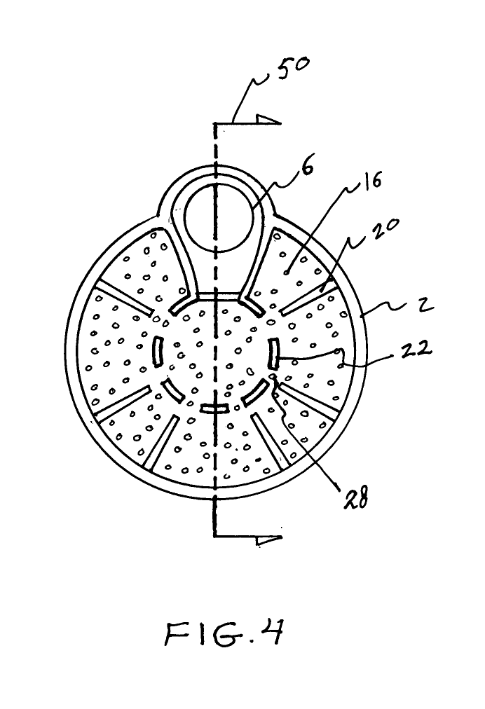

Referring now to FIG. 1 we see a perspective view of the invention 100. A front garment facing surface 2 forms a roughly circular convex shape. A cut out aperture 6 allows a portion of a liquid filled bag 4 to be available to the user. A rear wearer facing surface 8 is attached to the front facing surface 2 at the periphery 18. Both the front and rear facing surfaces 2, 8 are preferably constructed of thin walled plastic such as polystyrene. The thin walls of the plastic surface 2 and surface 8 are approximately twenty thousandths of an inch allowing the entire assembly 100 to flex when being worn within a woman's brassier as shown in FIG. 4.

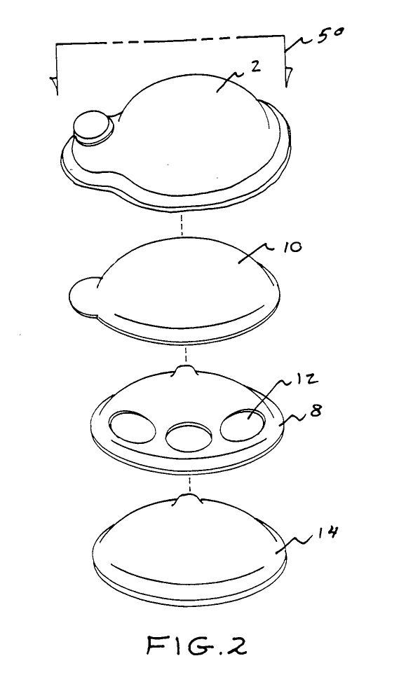

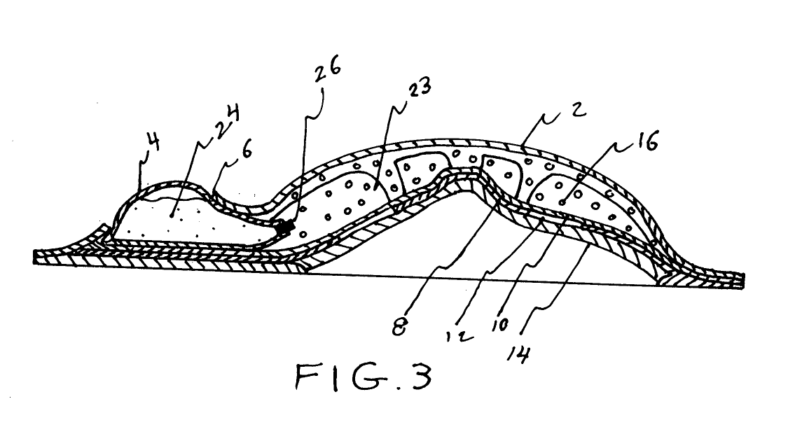

FIG. 2 shows an exploded view of the invention. Liquid 24 and powder 16 chemicals are held under garment facing surface 2 as will be described fully in FIG. 3. A very thin plastic film layer 10 holds the powder and liquid in place in a water tight manner. Rear wearer facing surface 8 includes apertures 12 that allow maximum coolness of the endothermic reaction to be transferred through the thin film 10 to the user's breast area. Absorbent pad 14 is adhered to the outside wall of the rear wearer facing surface 8. This absorbent pad 14 allows excess breast milk to be absorbed during use.

FIG. 3 shows a side section view of the invention as defined by section line 50 shown in FIGS. 2 and 4. Wearer facing surface 8 includes an indentation to allow room for the user's nipple. A flexible bag 4 contains a chemical liquid 24. The interior space between the garment facing surface and the wearer wearing surface contains a chemical powder. When the user depresses bag 4, it causes cork member 26 to pop, thereby expelling the liquid out where it mixes with powder 16. This mixing causes an endothermic cooling reaction. The cooling influence transfers through the thin film 10 and out of apertures 12 in wearer facing surface 8. The very thin plastic film layer 10 prevents the liquid from escaping the entire assembly. Ribs 23 keep powder 16 evenly dispersed, which helps create an even cooling effect. The ribs 22, 23 can be seen more clearly in the plan view shown in FIG. 4. This view shows the garment facing surface 2 from the inside. The liquid filled bag 4 has been removed for clarity purposes. The spaces between the ribs 28 allow the liquid 24 to flow from one chamber to another thereby completing the endothermic reaction by soaking the evenly dispersed powder 16.

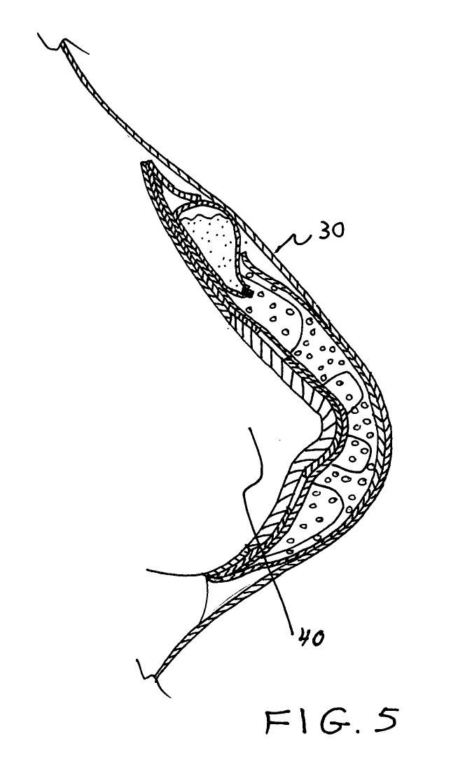

FIG. 5 shows a side section view of the invention 100 placed on user's breast 40 and nipple and held in place by the user's brassier 30. In this way the user can maintain the soothing and cooling effect of the invention while in public spaces.

I claim:

1. Nursing breast pad, comprising a front garment facing surface and a rear wearer facing surface attached to each other by a peripheral flange, wherein the space between said front facing surface and said rear facing surface contains a centrally located chemical endothermic cooling means.

2. The nursing breast pad of claim 1, wherein an absorbent pad is attached to the outside of said rear wearer facing surface.

3. The nursing breast pad of claim 1, wherein said breast a pad and said absorbent pad have a circular shape.

4. The nursing breast pad of claim 2, wherein the front and rear facing surfaces are made of thin walled plastic.

6. The nursing breast pad of claim 1, wherein said garment facing surface includes a convex shaped surface.

7. The nursing breast pad of claim 1, wherein the said endothermic chemicals are comprised of a liquid chemical which is contained within a flexible bag and a powder chemical which surrounds said bag;
said liquid containing bag capable of being pinched and burst by the user so that said liquid can mix with said powder to create said endothermic reaction.

8. The nursing breast pad of claim 2, wherein the outer surface of said absorbent surface is made of a soft, absorbent wick like cotton.

9. The nursing breast pad of claim 1, wherein the wearer facing surface is in permanent contact with the woman's breast when the pad is placed on the users brassiere.

10. The nursing breast pad of claim 1, wherein the cooling means is in permanent contact with the wearer's nipple when the pad is placed on the user's brassiere.

11. The nursing breast pad of claim 1, wherein the cooling means has a general rounded shape capable of facilitating it's placing on the inner surface of the wearer's brassiere.

12. The nursing breast pad of claim 2, wherein the annular absorbent surface is in full contact with the surface that surrounds the nipple of wearer's breast.

13. The nursing breast pad of claim 1, wherein the wearer is a breastfeeding mother.

14. The nursing breast pad of claim 13, wherein the cooling means is capable of soothing the nipples, alleviating pain and stopping bleeding.

15. The nursing breast pad of claim 13, wherein the absorbent surface is capable of providing absorbency of leaking breast milk.

16. The nursing breast pad of claim 1, wherein the diameter of the cooling means is larger than the regular diameter of a woman's nipple.

17. The nursing breast pad of claim 2, wherein the diameter of the absorbent surface is larger than the diameter of the cooling means.

18. The nursing breast pad of claim 1 wherein the said garment facing surface includes on its inside wall a plurality of ribs helping said powder to remain evenly dispersed.
